SEQUENCE LISTING (40 sequences)

SEQ ID NO: 1 - DNA, 20 bp, synthetic construct
  source 1..20: mol_type = other DNA
ttgatgtcgtgtatagtcga

SEQ ID NO: 2 - DNA, 20 bp, synthetic construct
  source 1..20: mol_type = other DNA
cagctggagcaaccgatact

SEQ ID NO: 3 - DNA, 19 bp, synthetic construct
  source 1..19: mol_type = other DNA
gcgattggtgaccagatcc

SEQ ID NO: 4 - DNA, 4950 bp, synthetic construct
  source 1..4950: mol_type = other DNA
ggtaccgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgatgtcgtgtatagtcgagttttagagctagaaatagcaagttaaaataaggctagtccgttatcaacttgaaaaagtggcaccgagtcggtgctttttttaaagaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgagggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 5 - DNA, 4950 bp, synthetic construct
  source 1..4950: mol_type = other DNA
ggtaccgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagctggagcaaccgatactgttttagagctagaaatagcaagttaaaataaggctagtccgttatcaacttgaaaaagtggcaccgagtcggtgctttttttaaagaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgagggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 6 - DNA, 4949 bp, synthetic construct
  source 1..4949: mol_type = other DNA
ggtaccgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcgattggtgaccagatccgttttagagctagaaatagcaagttaaaataaggctagtccgttatcaacttgaaaaagtggcaccgagtcggtgctttttttaaagaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgagggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 7 - DNA, 22 bp, synthetic construct
  source 1..22: mol_type = other DNA
gttagcttgtagttcccacttg

SEQ ID NO: 8 - DNA, 20 bp, synthetic construct
  source 1..20: mol_type = other DNA
gtgatacagccaggcagaag

SEQ ID NO: 9 - DNA, 20 bp, synthetic construct
  source 1..20: mol_type = other DNA
gtgatacagccaggcagaag

SEQ ID NO: 10 - DNA, 20 bp, synthetic construct
  source 1..20: mol_type = other DNA
gtgatacagccaggcagaag

SEQ ID NO: 11 - DNA, 22 bp, synthetic construct
  source 1..22: mol_type = other DNA
ctgtagtatgaaatcatccctg

SEQ ID NO: 12 - DNA, 18 bp, synthetic construct
  source 1..18: mol_type = other DNA
tcactttgccttcatcct

SEQ ID NO: 13 - DNA, 18 bp, synthetic construct
  source 1..18: mol_type = other DNA
tggtagagttgccaggac

SEQ ID NO: 14 - DNA, 18 bp, synthetic construct
  source 1..18: mol_type = other DNA
cctgtaattgtggcttcc

SEQ ID NO: 15 - DNA, 19 bp, synthetic construct
  source 1..19: mol_type = other DNA
ggatgtgctcttgtgggtc

SEQ ID NO: 16 - DNA, 20 bp, synthetic construct
  source 1..20: mol_type = other DNA
gcatctgtaaccgacaccac

SEQ ID NO: 17 - DNA, 18 bp, synthetic construct
  source 1..18: mol_type = other DNA
gcattcacccgctcagta

SEQ ID NO: 18 - DNA, 18 bp, synthetic construct
  source 1..18: mol_type = other DNA
cggctgtggcaacttact

SEQ ID NO: 19 - DNA, 20 bp, synthetic construct
  source 1..20: mol_type = other DNA
tgtaggttctttcctgggta

SEQ ID NO: 20 - DNA, 18 bp, synthetic construct
  source 1..18: mol_type = other DNA
agcaatgccactaacagg

SEQ ID NO: 21 - DNA, 19 bp, synthetic construct
  source 1..19: mol_type = other DNA
tccgaagcctgagaagacc

SEQ ID NO: 22 - DNA, 19 bp, synthetic construct
  source 1..19: mol_type = other DNA
gcagcagggagacagttca

SEQ ID NO: 23 - DNA, 18 bp, synthetic construct
  source 1..18: mol_type = other DNA
cctgaccagcctgtttga

SEQ ID NO: 24 - DNA, 20 bp, synthetic construct
  source 1..20: mol_type = other DNA
cataataggagttcccagtc

SEQ ID NO: 25 - DNA, 18 bp, synthetic construct
  source 1..18: mol_type = other DNA
aagcctgggactccttag

SEQ ID NO: 26 - DNA, 18 bp, synthetic construct
  source 1..18: mol_type = other DNA
cttgcgtgggtttaatgt

SEQ ID NO: 27 - DNA, 18 bp, synthetic construct
  source 1..18: mol_type = other DNA
atctgctgagccacaatg

SEQ ID NO: 28 - DNA, 18 bp, synthetic construct
  source 1..18: mol_type = other DNA
tcaatagcagcccaacct

SEQ ID NO: 29 - DNA, 19 bp, synthetic construct
  source 1..19: mol_type = other DNA
attggctttgaaacactcc

SEQ ID NO: 30 - DNA, 18 bp, synthetic construct
  source 1..18: mol_type = other DNA
tgctgtgagccgaggtat

SEQ ID NO: 31 - DNA, 19 bp, synthetic construct
  source 1..19: mol_type = other DNA
ctggcattgtagcctgacc

SEQ ID NO: 32 - DNA, 20 bp, synthetic construct
  source 1..20: mol_type = other DNA
ggaaggcagttactcaaaga

SEQ ID NO: 33 - DNA, 20 bp, synthetic construct
  source 1..20: mol_type = other DNA
gggcaggaggacaaggagtt

SEQ ID NO: 34 - DNA, 20 bp, synthetic construct
  source 1..20: mol_type = other DNA
tgcagcgaggacctgaatgg

SEQ ID NO: 35 - DNA, 20 bp, synthetic construct
  source 1..20: mol_type = other DNA
accatcactccgggcgactc

SEQ ID NO: 36 - DNA, 20 bp, synthetic construct
  source 1..20: mol_type = other DNA
catgtgggacctcggattga

SEQ ID NO: 37 - DNA, 20 bp, synthetic construct
  source 1..20: mol_type = other DNA
ggtcttgactgggagaaaga

SEQ ID NO: 38 - DNA, 19 bp, synthetic construct
  source 1..19: mol_type = other DNA
tcataaagcacctcttcac

SEQ ID NO: 39 - DNA, 20 bp, synthetic construct
  source 1..20: mol_type = other DNA
agctgcggtcccggtcactc

SEQ ID NO: 40 - DNA, 44 bp, Sus scrofa
  source 1..44: mol_type = other DNA
gtcattccattgatgtcgtgtatagtcgagggaaaagtatgggt

(19) United States
(12) Patent Application Publication
(10) Pub. No.: US 2024/0276958 A1
(43) Pub. Date: Aug. 22, 2024

(54) SGRNA AND CONSTRUCTING DUAL PIG MODEL OF SEVERE IMMUNODEFICIENCY AND LIVER INJURY AND USE THEREOF

(71) Applicant: Yunnan Agricultural University, Kunming (CN)

(72) Inventors: Hong-jiang Wei, Kunming (CN); Qing-feng Chen, Kunming (CN); Heng Zhao, Kunming (CN); De-ling Jiao, Kunming (CN); Hong-ye Zhao, Kunming (CN)

(21) Appl. No.: 18/646,802

(22) Filed: Apr. 26, 2024

Related U.S. Application Data
(63) Continuation-in-part of application No. PCT/CN2021/128723, filed on Nov. 4, 2021.

(51) Int. Cl. A01K 67/0276; C07K 14/715; C12N 9/22; C12N 15/11; C12N 15/90; C12Q 1/6883

(52) U.S. Cl. CPC A01K 67/0276; C12N 9/22; C12N 15/11; C12N 15/907; C12Q 1/6883; A01K 2217/15; A01K 2227/108; A01K 2267/0387; C07K 14/7155; C12Q 2600/124

(57) ABSTRACT

The present disclosure relates to a sgRNA and constructing a dual pig model of severe immunodeficiency and liver injury and use thereof. The method comprises the steps of knocking out RAG2, IL2Rγ and FAH genes in a porcine fetal fibroblast by using a CRISPR/Cas9 technology, constructing a RAG2^{-/-}/IL2Rγ^{-/Y}/FAH^{-/-} triple-gene edited cloned pig by using a somatic cell nuclear transfer technology, and obtaining a dual pig model of severe immunodeficiency and liver injury through phenotypic analysis and identification. The method overcomes the problems of long production period, low efficiency, irreversible damage, unsatisfactory use in a humanization degree and the like in the existing model construction technology, can realize a batch construction of the dual pig model of severe immunodeficiency and liver injury by a continuous cloning technology, and has great advantages and potential market application prospects in the related fields of tumor biology, cell transplantation, humanized animal models and the like.

### CROSS-REFERENCE TO RELATED APPLICATIONS

This application is a continuation-in-part of International Application No. PCT/CN2021/128723, filed on Nov. 4, 2021, which is incorporated herein by reference in its entirety.

### REFERENCE TO SEQUENCE LISTING

This application includes a Sequence Listing filed electronically as an XML file named Sequence listing_RONDA-24001-USPT.xml, created on Apr. 26, 2024, with a size of 51,145 bytes. The Sequence Listing is incorporated herein by reference.

### TECHNICAL FIELD

The present disclosure belongs to the technical field of animal biology and specifically relates to a sgRNA and constructing a dual pig model of severe immunodeficiency and liver injury and use thereof.

### BACKGROUND

Immunodeficient animals play an important role in the fields of biomedical and basic medical research. Liver transplantation is an important treatment mode for treating end-stage liver failure. Liver donor shortage is a main bottleneck for restricting clinical application of liver transplantation. A hepatocyte transplantation technology is one of important ways for solving the problem of serious shortage of liver organs. Human hepatocytes are implanted into a dual animal model of immunodeficiency and liver injury to produce a humanized liver, and thus the humanized hepatocytes are obtained in batches so as to solve the problem of clinical primary hepatocyte source limitation. The hepatocyte transplantation technology plays an important role in the treatment of diseases caused by human hepatitis B virus (HBV) and hepatitis C virus (HCV), vaccine research and development, pharmacology, toxicology and the like.

At present, by using a dual mouse model of severe combined immunodeficiency disorder (SCID) and urokinase type plasminogen activator (uPA), a mouse, with a humanized liver, having the chimerism as high as 80%, is successfully obtained after 6 weeks of the implantation of human hepatocytes. Based on a FAH^{-/-}/Rag^{-/-}/IL2Rγ^{-/-} triple-gene modified mouse model, a large amount of humanized hepatocytes have been successfully amplified, and the liver regeneration rate can reach 80% or more. However, there are still many problems in the current mouse model:
(1) rodents such as mice and the like have far genetic relationship with human, large size difference and short service life, and the utilization of the rodents such as mice and the like is difficult to highly simulate the physiological metabolic process of human, such that the use of immunodeficient mice in the field of life science research is greatly limited;
(2) at present, a relatively traditional gene editing method is used for constructing the dual mouse model, and even chemical drugs are selected for establishing liver injury, irreversible injury is caused, and the success rate of molding is low, which is not beneficial to subsequent simulation of human to carry out surgical operation and preclinical evaluation;
(3) a FAH^{-/-}/Rag^{-/-}/IL2Rγ^{-/-} triple-gene simultaneously modified mouse model is currently commonly obtained by a hybridization mode: firstly, a Rag^{-/-}/IL2Rγ^{-/-} mouse is mated with a FAH^{-/-} mouse to generate a Rag^{-/+}/IL2Rγ^{-/+}/FAH^{-/+} individual, then the individuals are self-crossed to obtain a FAH^{-/-}/Rag^{-/-}/IL2Rγ^{-/-} triple-gene simultaneously modified individual, some chimeric mice are obtained by injecting fertilized eggs, the period constructed by the method is long, and the model production efficiency is low; and
(4) at present, in a FAH^{-/-}/Rag^{-/-}/IL2Rγ^{-/-} mouse model, NK cells cannot be removed, inflammatory reaction is different from that of a human body, and thus a human model cannot be well remodeled.

Pigs are large mammals, and very similar to humans in genome homology, body size, physiological and biochemical indexes, tissue dissection, immune metabolism and the like, such that the establishment of a dual pig model of immunodeficiency and liver injury can be better used in the research of relevant fields of human tumor biology, cell transplantation, immunodeficient models and the like, and has important significance for promoting the rapid development of life science industry.

### SUMMARY

Aiming at the technical problems and the actual needs, the present disclosure provides a sgRNA and constructing a dual pig model of severe immunodeficiency and liver injury and use thereof. On the basis of a CRISPR/Cas9 gene editing technology, the method simultaneously knock out RAG2, IL2Rγ and FAH genes in a porcine fetal fibroblast to obtain a RAG2^{-/-}/IL2Rγ^{-/Y}/FAH^{-/-} triple-gene edited porcine fibroblast cell line, which is used as a donor cell to carry out somatic cell nuclear transfer to construct a RAG2^{-/-}/IL2Rγ^{-/Y}/FAH^{-/-} triple-gene edited cloned pig, and the pathological, immunological, cytological and liver function detection and phenotypic analysis and identification of the cloned pig are further carried out to obtain a dual pig model of severe immunodeficiency and liver injury. The model shows immunodeficiency, liver injury, thymus and spleen dysplasia, reduced mature T cell number, deficiency of B cells and NK cells, and has great advantages and a wide market prospect in the related fields of tumor biology, cell transplantation, humanized animal models and the like.

To realize the above purposes, the present disclosure is realized through the following technical solutions:

The present disclosure provides a RAG2/IL2Rγ/FAH triple-gene targeting vector, a recombinant plasmid for RAG2/IL2Rγ/FAH triple-gene editing of a cell, and a RAG2/IL2Rγ/FAH triple-gene knockout porcine fibroblast cell line.

The RAG2/IL2Rγ/FAH triple-gene targeting vector, wherein the targeting vector is an sgRNA expression vector based on a CRISPR/Cas9 system, and an sgRNA comprises a RAG2-sgRNA, an IL2Rγ-sgRNA, and an FAH-sgRNA; and the sgRNA acts on a site located in a coding region of a pig RAG2 gene, a 5th exon of an IL2Rγ gene, and a 2nd exon of an FAH gene.

Further, the nucleotide sequence of the RAG2-sgRNA is shown in SEQ ID NO: 1; the nucleotide sequence of the IL2Rγ-sgRNA is shown in SEQ ID NO: 2; and the nucleotide sequence of the FAH-sgRNA is shown in SEQ ID NO: 3.

Further, a framework vector is a pGL3-U6-sgRNA (Addgene no: 51133).

The recombinant plasmid for RAG2/IL2Rγ/FAH triple-gene editing of a cell, wherein the nucleotide sequence of the recombinant plasmid RAG2-sgRNA is shown in SEQ ID NO: 4; the nucleotide sequence of the recombinant plasmid IL2Rγ-sgRNA is shown in SEQ ID NO: 5; and the nucleotide sequence of the recombinant plasmid FAH-sgRNA is shown in SEQ ID NO: 6.

Further, the cell is a porcine fibroblast cell line.

The RAG2/IL2Rγ/FAH triple-gene knockout porcine fibroblast cell line, wherein the targeting vector or the recombinant plasmid is transfected into the porcine fetal fibroblast cell line to obtain a targeted positive cell clone, that is the RAG2/IL2Rγ/FAH triple-gene knockout porcine fibroblast cell line or a RAG2^{-/-}/IL2Rγ^{-/Y}/FAH^{-/-} triple-gene edited porcine fibroblast cell line.

The present disclosure further provides a method for constructing a dual pig model of severe immunodeficiency and liver injury by using a CRISPR/Cas9 technology. The method uses the CRISPR/Cas9 gene editing technology to knock out RAG2, IL2Rγ and FAH genes in a porcine fetal fibroblast, and uses a somatic cell nuclear transfer technology to construct a RAG2^{-/-}/IL2Rγ^{-/Y}/FAH^{-/-} triple-gene edited cloned pig. The method comprises the following specific steps:
1) construction of RAG2^{-/-}/IL2Rγ^{-/Y}/FAH^{-/-} triple-gene mutation expression vector on the basis of CRISPR/Cas9 system
aiming at a coding region of a pig RAG2 gene (Gene ID: 100151744), a 5th exon of an IL2Rγ gene (Gene ID: 397156), and a 2nd exon of an FAH gene (Gene ID: 100623036), designing a corresponding sgRNA targeting vector and connecting same to a framework vector to obtain a pGL3-U6-sgRNA recombinant plasmid;
2) screening of RAG2^{-/-}/IL2Rγ^{-/Y}/FAH^{-/-} triple-gene edited porcine fibroblast cell line
co-transfecting the pGL3-U6-sgRNA recombinant plasmid and a pST1374-NLS-flag-linker-Cas9 (Addgene no: 44758) into a porcine fetal fibroblast cell, and obtaining a RAG2^{-/-}/IL2Rγ^{-/Y}/FAH^{-/-} triple-gene edited porcine fibroblast cell line through single cell clone genotype identification and screening;
3) somatic cell nuclear transfer and embryo transfer
performing a somatic cell nuclear transfer by using the RAG2^{-/-}/IL2Rγ^{-/Y}/FAH^{-/-} triple-gene edited porcine fibroblast cell line as a donor cell to construct a RAG2^{-/-}/IL2Rγ^{-/-}/FAH^{-/-} triple-gene edited pig cloned embryo, further transplanting the cloned embryo into an oviduct of a surrogate sow, and delivering a RAG2^{-/-}/IL2Rγ^{-/Y}/FAH^{-/-} triple-gene edited cloned pig after 114 days of pregnancy; and
4) genotype identification and phenotypic analysis of cloned piglet
extracting genomic DNA from a cloned piglet, identifying a RAG2/IL2Rγ/FAH genotype of the cloned piglet by using a molecular biology method, further performing histological and immunohistochemical analysis on organs of thymus, spleen, liver and the like of the cloned pig, and performing immunological and cytological analysis and identification on a mature T cell, a B cell, an NK cell, a hepatocyte and the like to obtain a RAG2^{-/-}/IL2Rγ^{-/Y}/FAH^{-/-} triple-gene edited dual pig model of severe immunodeficiency and liver injury.

Further, in step 1), the sgRNA comprises a RAG2-sgRNA, an IL2Rγ-sgRNA, and an FAH-sgRNA; wherein the nucleotide sequence of the RAG2-sgRNA is shown in SEQ ID NO: 1; the nucleotide sequence of the IL2Rγ-sgRNA is shown in SEQ ID NO: 2; and the nucleotide sequence of the FAH-sgRNA is shown in SEQ ID NO: 3; and the vector is a framework vector pGL3-U6-sgRNA (Addgene no: 51133).

Further, in step 2), the transfection method comprises lipofection transfection and/or nuclear transfection, preferably nuclear transfection.

Further, in step 3), the donor cell can be a RAG2^{-/-}/IL2Rγ^{-/Y}/FAH^{-/-} triple-gene edited single cell clone or a cloned fetal fibroblast and a cloned porcine fibroblast cell.

Further, in step 3), the transfection amount ratio of the pST1374-NLS-flag-linker-Cas9 plasmid and the RAG2/IL2Rγ/FAH-sgRNA=2:1.

Further, in step 3), if batch production of the RAG2^{-/-}/IL2Rγ^{-/Y}/FAH^{-/-} triple-gene edited cloned pig is required, the pig can be obtained by a continuous cloning technology.

Use of the targeting vector, the recombinant plasmid, the cell or the method in constructing a dual pig model of severe immunodeficiency and liver injury and in the fields of tumor biology, cell transplantation, humanized animal model research and the like.

### Beneficial Effects of the Present Disclosure:

1. The present disclosure constructs a RAG2^{-/-}/IL2Rγ^{-/Y}/FAH^{-/-} dual pig model of severe immunodeficiency and liver injury on the basis of a CRISPR/Cas9 gene editing technology. Compared with models of rodents such as mice and non-human primates, the pig model is more similar to humans in genome homology, body size, physiological and biochemical indexes, tissue dissection, immune metabolism and the like, has no ethical limitation, plays an important role in the field of human life science research, and has a huge biomedical research application prospect and market value.

2. The present disclosure firstly obtains the dual pig model of severe immunodeficiency and liver injury through the CRISPR/Cas9 technology. The model has a short production cycle and high production efficiency. Besides, after an FAH gene is knocked out, the reversibility can be realized by using an NTBC drug, and thus the problem of irreversible use of the existing model construction technology is solved.

3. In the process of constructing RAG2^{-/-}/IL2Rγ^{-/Y} immunodeficient mice in the prior art, since the immune regulation system of mice is different from that of humans, NK cells cannot be removed after the RAG2^{-/-}/IL2Rγ^{-/Y} knockout. In addition, RAG and IL2Rγ genes are required to be simultaneously edited when the NK cells are removed. Pluralities of genes are knocked out by a zinc finger nuclease and a TALENs technology conventionally, which has the low efficiency. The present disclosure overcomes the efficiency problem of multigene editing through the CRISPR/Cas9 technology. The RAG2^{-/-}/IL2Rγ^{-/Y}/FAH^{-/-} model pig of the present disclosure solves the problem of effectively removing NK cells in peripheral blood, reduces the occurrence of inflammatory reaction to a certain extent, has a higher humanization degree, and effectively reduces immunological rejection to a certain extent.

4. The present disclosure provides sgRNAs suitable for editing three genes of pig FAH, RAG2 and IL2Rγ through activity screening of different sgRNAs and off-target detection, verifies the functions of edited regions after being knocked out, and solves the problems of off-target and low birth efficiency of a cloned animal after polygene modification in the CRISPR/Cas9 gene editing technology.

5. The RAG2^{-/-}/IL2Rγ^{-/Y}/FAH^{-/-} triple-gene edited cloned pig obtained by the present disclosure shows immunodeficiency, severe liver injury, thymus and spleen dysplasia, reduced mature T cell number, deficiency of B cells and NK cells, high apoptosis rate of hepatocytes, and high immunodeficient degree. The batch production of a dual pig model of severe immunodeficiency and liver injury can be realized by a continuous cloning technology. The construction of the RAG2^{-/-}/IL2Rγ^{-/Y}/FAH^{-/-} triple-gene edited dual pig model of severe immunodeficiency and liver injury has great advantages and potential market application prospects in the related fields of hepatocyte transplantation technology, tumor biology, cell transplantation, humanized animal models and the like.

### BRIEF DESCRIPTION OF THE DRAWINGS

FIG. 1 is a schematic diagram of the construction of a RAG2^{-/-}/IL2Rγ^{-/Y}/FAH^{-/-} triple-gene knockout cloned pig;

FIG. 2 shows a schematic diagram and identification result of targeting of a RAG2^{-/-} gene-edited porcine fetal fibroblast cell line, wherein (A) shows a schematic diagram of a RAG2 gene targeting strategy, (B) shows a electrophoresis result of a RAG2 gene PCR amplification products, C1˜C9 is clone site No. 1˜No. 9, M is Marker, Ctrl is a control group, (C) shows a Sanger sequencing result of a RAG2 gene knockout No. 9 single cell clone, wherein RAG2 gene is shown as SEQ ID NO: 40, (D) shows a RAG2 gene knockout cloned porcine fetus, (E) shows a T7EN1 digestion identification results of a RAG2 gene knockout cloned porcine fetal PCR products, F1˜F5 is fetus No. 1˜No. 5, M is Marker, Ctrl is a control group;

FIG. 3 shows a screening result of activities of targeting sgRNAs of an IL2Rγ gene and an FAH gene, wherein A of FIG. 3 shows a detection diagram of an FAH gene targeting sgRNA1/2/3 cleavage efficiency, B of FIG. 3 shows a detection diagram of an IL2Rγ gene targeting sgRNA1/2 cleavage efficiency, C of FIG. 3 shows a quantification result of an FAH and IL2Rγ targeting sgRNA cleavage efficiency, D of FIG. 3 shows an FAH and IL2Rγ targeting sgRNA nucleotide series, wherein FAH-sg1, SEQ ID NO: 37; FAH-sg2, SEQ ID NO: 3; FAH-sg3, SEQ ID NO: 38; IL2RG-sg1, SEQ ID NO: 39; IL2RG-sg2, SEQ ID NO: 2;

FIG. 4 shows a schematic diagram and identification result of targeting of a RAG2^{-/-}/IL2Rγ^{-/Y}/FAH^{-/-} triple-gene edited porcine fetal fibroblast cell line, wherein A of FIG. 4 shows a schematic diagram of an IL2Rγ and FAH gene targeting strategy, B of FIG. 4 shows a PCR identification result of an IL2Rγ and FAH gene knockout single cell clone, C24˜C39 is clone site No. 24˜No. 39, M is Marker, Ctrl is a control group, C of FIG. 4 shows a Sanger sequencing result of an IL2Rγ gene knockout No. 25 single cell clone, and D of FIG. 4 shows a Sanger sequencing result of an FAH gene knockout No. 25 single cell clone;

FIG. 5 shows a RAG2^{-/-}/IL2Rγ^{-/Y}/FAH^{-/-} triple-gene edited cloned pig;

FIG. 6 shows a genotype identification result of the RAG2^{-/-}/IL2Rγ^{-/Y}/FAH^{-/-} triple-gene edited cloned pig, wherein A of FIG. 4 shows a PCR identification result of a RAG2, IL2Rγ and FAH gene knockout in cloned pigs No. 19 and No. 20 (RGFP19 indicates cloned pig No. 19, RGFP20 indicates cloned pig No. 20, WT is a control pig without gene editing), B of FIG. 5 shows a Sanger sequencing result of a RAG2, IL2Rγ and FAH gene knockout in cloned pigs No. 19 and No. 20 (P19 indicates cloned pig No. 19, P20 indicates cloned pig No. 20, WT is a control pig without gene editing);

FIG. 7 shows an immune function deficiency identification result of the RAG2^{-/-}/IL2Rγ^{-/Y}/FAH^{-/-} triple-gene edited cloned pig, wherein A of FIG. 7 is a survival curve of the RAG2^{-/-}/IL2Rγ^{-/Y}/FAH^{-/-} triple-gene edited cloned pig; B of FIG. 7 shows thymus anatomical drawings of the RAG2^{-/-}/IL2Rγ^{-/Y}/FAH^{-/-} triple-gene edited cloned pig; C of FIG. 7 shows spleen anatomical drawings of the RAG2^{-/-}/IL2Rγ^{-/Y}/FAH^{-/-} triple-gene edited cloned pig; D of FIG. 7 shows a HE staining result of the spleen of the RAG2^{-/-}/IL2Rγ^{-/Y}/FAH^{-/-} triple-gene edited cloned pig; (E-G) of FIG. 7 show results of detection of mature T cells (labeled with a CD3 antibody), B cells (labeled with a CD45RA antibody), and NK cells (labeled with CD3 and CD16 antibodies) in peripheral blood or spleen of the RAG2^{-/-}/IL2Rγ^{-/Y}/FAH^{-/-} triple-gene edited cloned pig; H of FIG. 7 shows mRNA expression levels of CD4, CD8, IL2Rγ and CD19 genes in the RAG2^{-/-}/IL2Rγ^{-/Y}/FAH^{-/-} triple-gene edited cloned spleen; I of FIG. 7 shows levels of proteins expressed by the IL2Rγ gene of the RAG2^{-/-}/IL2Rγ^{-/Y}/FAH^{-/-} triple-gene edited cloned pig; and J of FIG. 7 shows a rearrangement result of V(D)J of the RAG2^{-/-}/IL2Rγ^{-/Y}/FAH^{-/-} triple-gene edited cloned pig; and

FIG. 8 shows a result of phenotypic testing of liver injury of the RAG2^{-/-}/IL2Rγ^{-/Y}/FAH^{-/-} triple-gene edited cloned pig, wherein A of FIG. 8 shows a schematic diagram of an NTBC dosage regimen for the RAG2^{-/-}/IL2Rγ^{-/Y}/FAH^{-/-} triple-gene edited cloned pig; B of FIG. 8 shows an FAH immunohistochemical detection result of the liver of the RAG2^{-/-}/IL2Rγ^{-/Y}/FAH^{-/-} triple-gene edited cloned pig; C of FIG. 8 shows levels of proteins expressed by the FAH gene in the liver of the RAG2^{-/-}/IL2Rγ^{-/Y}/FAH^{-/-} triple-gene edited cloned pig; D of FIG. 8 shows levels of proteins expressed by the FAH gene in the testes of the RAG2^{-/-}/IL2Rγ^{-/Y}/FAH^{-/-} triple-gene edited cloned pig; E of FIG. 8 shows an HE staining result of the liver of the RAG2^{-/-}/IL2Rγ^{-/Y}/FAH^{-/-} triple-gene edited cloned pig; F of FIG. 8 shows an apoptosis detection result of hepatocytes of the RAG2^{-/-}/IL2Rγ^{-/Y}/FAH^{-/-} triple-gene edited cloned pig; G of FIG. 8 shows an apoptosis quantification result of the RAG2^{-/-}/IL2Rγ^{-/Y}/FAH^{-/-} triple-gene edited cloned pig; and H of FIG. 8 shows mRNA expression levels of apoptosis related genes BID, BAX, PUMA and BCL2L1 of the liver of the RAG2^{-/-}/IL2Rγ^{-/Y}/FAH^{-/-} triple-gene edited cloned pig.

### DETAILED DESCRIPTION OF THE EMBODIMENTS

In order to make the purposes, technical solutions and beneficial effects of the present disclosure more apparent, preferred examples of the present disclosure will be described in detail below to facilitate understanding of those skilled in the art.

### Example 1: Construction of Pig RAG2^{-/-}/IL2Rγ^{-/Y}FAH^{-/-} Triple-Gene Targeting Vector

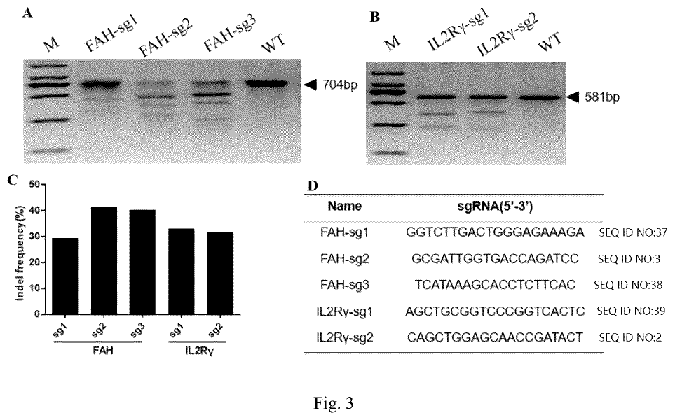

The nucleotide sequences of a pig RAG2 gene (Gene ID: 100151744), an IL2Rγ gene (Gene ID: 397156), and an FAH gene (Gene ID: 100623036) were searched in the NCBI database. Aiming at a coding region of the pig RAG2 gene, a 5th exon sequence of the IL2Rγ gene, and a 2nd exon sequence of the FAH gene, targeting gRNA sites were designed and screened by using an online software (http://crispor.tefor.net/), the sgRNAs were ligated to a framework vector to obtain a RAG2-gRNA (SEQ ID NO: 1) targeting vector (FIG. 2), and the optimal pig IL2Rγ/FAH gene recombination targeting vectors were further screened to obtain an IL2Rγ-gRNA (SEQ ID NO: 2) and an FAH-gRNA (SEQ ID NO: 3) respectively (FIG. 3, The sequences numbers of other sgRNAs are: FAH-sg1, SEQ ID NO: 37; FAH-sg2, SEQ ID NO: 38; FAH-sg3, SEQ ID NO: 39; IL2Rγ-sg1, SEQ ID NO: 40; IL2Rγ-sg2, SEQ ID NO: 41).

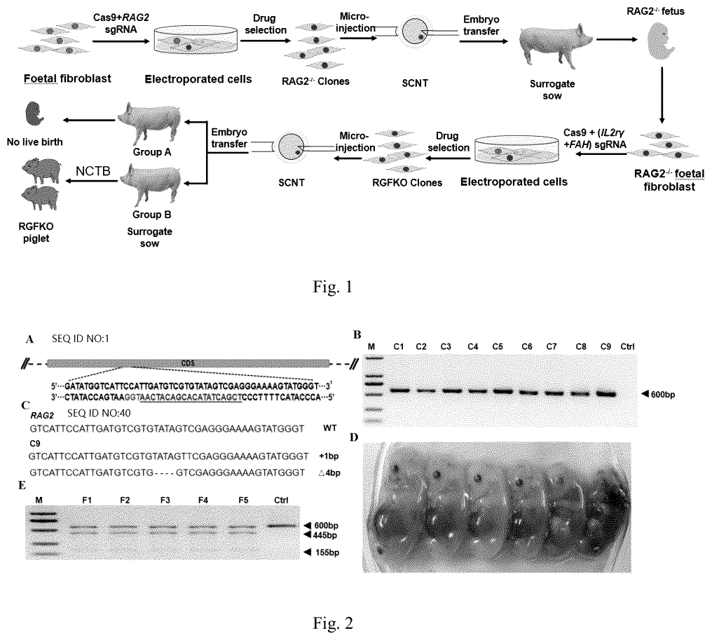

The targeting vectors were co-transfected into a porcine fetal fibroblast, and a RAG2^{-/-}/IL2Rγ^{-/Y}/FAH^{-/-} triple-gene mutant porcine fetal fibroblast cell line was obtained by screening and was used as a donor cell for somatic cell nuclear transfer to construct a RAG2^{-/-}/IL2Rγ^{-/Y}/FAH^{-/-} triple-gene edited cloned pig (FIG. 1 is a schematic diagram of the construction of a RAG2^{-/-}IL2Rγ^{-/Y}/FAH^{-/-} triple-gene knockout cloned pig, which is described in detail below).

### Example 2: Construction of RAG2^{-/-}/IL2Rγ^{-/Y}/FAH^{-/-} Triple-Gene Edited Cloned Pig

1. Aiming at the coding region of the pig RAG2 gene (Gene ID: 100151744), the 5th exon sequence of the IL2Rγ gene (Gene ID: 397156), and the 2nd exon sequence of the FAH gene (Gene ID: 100623036), targeting gRNA sites, the RAG2-gRNA (SEQ ID NO: 1), the IL2Rγ-gRNA (SEQ ID NO: 2), and the FAH-gRNA (SEQ ID NO: 3), respectively, were designed and screened.

2. The gRNA sequence SEQ ID NO: 1 of the RAG2 gene was ligated to a GL3-U6-sgRNA framework vector, a recombinant plasmid RAG2-GL3-U6-sgRNA with a correct sequence after sequence verification and a pST1374-NLS-flag-linker-Cas9 plasmid (pST1374-NLS-flag-linker-Cas 9:RAG2-GL3-U6-sgRNA=2:1) were co-transfected into a porcine fetal fibroblast by a nuclear transfection instrument, and 9 single cell clones were obtained by screening, wherein No. 9 single cell clone was RAG2 double allele knockout, named RAG2KO-09, further the RAG2KO-09 single cell clone was used as a donor cell for somatic cell nuclear transfer, a cloned embryo was transferred into a surrogate sow, 6 RAG2KO fetuses were obtained after 35 days, and RAG2KO porcine fetal fibroblast cell lines were isolated (FIG. 2).

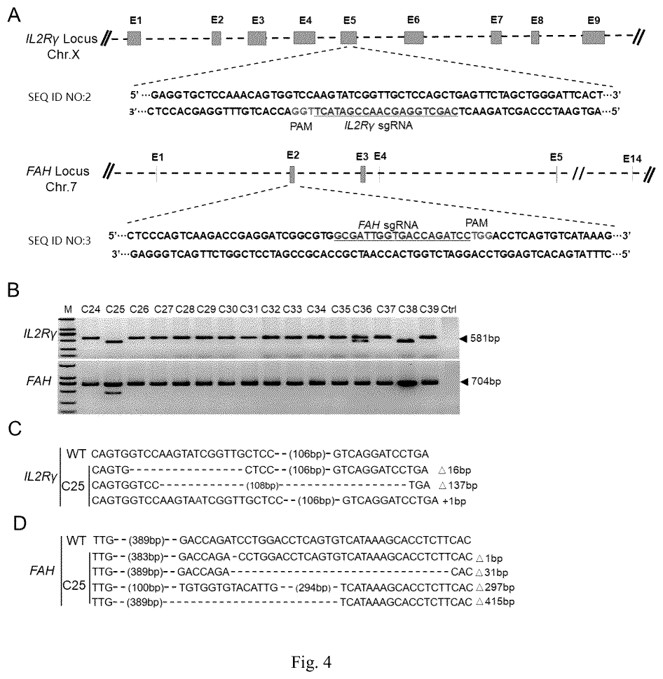

3. Further, the gRNA sequences SEQ ID NO:2 and SEQ ID NO:3 corresponding to IL2Rγ and FAH genes were respectively ligated to the GL3-U6-sgRNA framework vector, the recombinant plasmids IL2Rγ-GL3-U6-sgRNA and FAH-GL3-U6-sgRNA were obtained by correct sequencing sequences and these two recombinant plasmids and the pST1374-NLS-flag-linker-Cas9 plasmid were co-transfected to the RAG2KO porcine fetal fibroblast through the nuclear transfection instrument to obtain 39 single cell clones in total, and it was screened and identified that No. 25 single clone genotype was a RAG2^{-/-}/IL2Rγ^{-/Y}/FAH^{-/-} triple-gene mutant clone (FIG. 4).

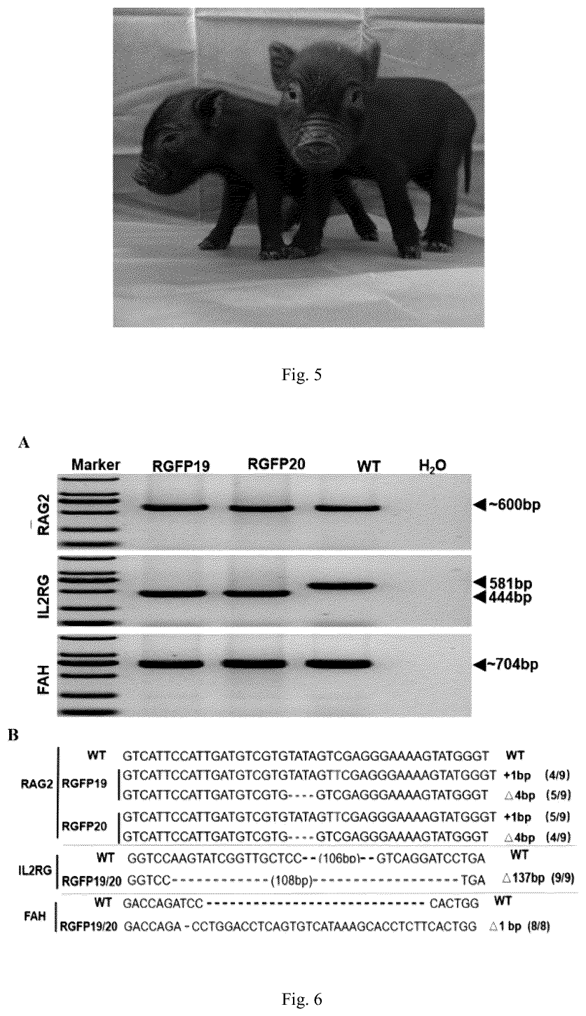

4. The No. 25 RAG2^{-/-}/IL2Rγ^{-/Y}/FAH^{-/-} triple-gene edited single cell clone screened in step (3) was used as a donor cell for somatic cell nuclear transfer, a cloned embryo was transferred into the uterus of an oestrous surrogate sow, and 2 alive cloned piglets were obtained after 114 days of pregnancy (FIG. 5).

### Example 3: Genotype Identification and Phenotypic Analysis of RAG2^{-/-}/IL2Rγ^{-/Y}/FAH^{-/-} Triple-Gene Edited Cloned Pig

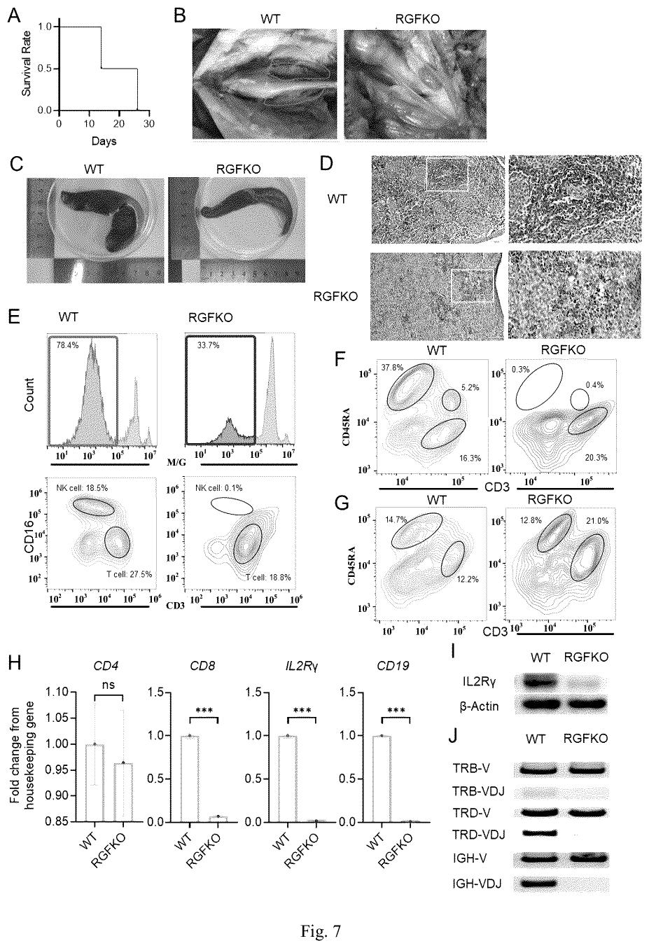

Genotype identification: genomic DNA of the 2 alive cloned piglets (RGFP19 and RGFP20) was extracted, the mutation of the RAG2/IL2Rγ/FAH genes of the cloned pig was identified by PCR, T7ENI, Sanger sequencing and the like. The results showed that the pigs were both RAG2^{-/-}/IL2Rγ^{-/Y}/FAH^{-/-} triple-gene knockout type (FIG. 6), the off-target of the RAG2^{-/-}/IL2Rγ^{-/Y}/FAH^{-/-} triple-gene edited cloned pig was further detected, and no off-target was found (Table 1).

TABLE 1
Off-target detection results of RAG2^{-/-}/IL2Rγ^{-/Y}/FAH^{-/-}triple-gene edited
cloned pig

| | | | | | Forward | Reverse | |
|---|---|---|---|---|---|---|---|
| Name | Gene | Chromosome | Position | Strand | Primer (5′-3′) | Primer (5′-3′) | off target |
| OTS- | RAG2 | Chr13 | 55792653 | − | GTTAGCTTGT | GTGATACAGC | − |
| R1 | | CM000824.5 | | | AGTTCCCACT | CAGGCAGAA | |
| | | | | | TG (SEQ ID | G (SEQ ID | |
| | | | | | NO: 7) | NO: 8) | |
| OTS- | | Chr13 | 161374794 | − | GTGATACAGC | GTGATACAGC | − |
| R2 | | CM000824.5 | | | CAGGCAGAA | CAGGCAGAA | |
| | | | | | G (SEQ ID | G (SEQ ID | |
| | | | | | NO: 9) | NO: 10) | |
| OTS- | | Chr13 | 161601500 | − | CTGTAGTATG | TCACTTTGCC | − |
| R3 | | CM000824.5 | | | AAATCATCCC | TTCATCCT (SEQ | |
| | | | | | TG (SEQ ID | ID NO: 12) | |
| | | | | | NO: 11) | | |
| OTS- | | Chr13 | 161881417 | − | TGGTAGAGTT | CCTGTAATTG | − |
| R4 | | CM000824.5 | | | GCCAGGAC | TGGCTTCC | |
| | | | | | (SEQ ID | (SEQ ID | |
| | | | | | NO: 13) | NO: 14) | |
| OTS- | IL2Rγ | CM000815.5 | 2450609 | + | GGATGTGCTC | GCATCTGTAA | − |
| G1 | | Chr4 | | | TTGTGGGTC | CCGACACCAC | |
| | | | | | (SEQ ID | (SEQ ID | |
| | | | | | NO: 15) | NO: 16) | |
| OTS- | | CM000821.5 | 10005709 | − | GCATTCACCC | CGGCTGTGGC | − |
| G2 | | Chr10 | | | GCTCAGTA | AACTTACT | |
| | | | | | (SEQ ID | (SEQ ID | |
| | | | | | NO: 17) | NO: 18) | |
| OTS- | FAH | CM000812.5 | 195936006 | + | TGTAGGTTCT | AGCAATGCC | − |
| F1 | | Chr1 | | | TTCCTGGGTA | ACTAACAGG | |
| | | | | | (SEQ ID | SEQ ID | |
| | | | | | NO: 19) | (NO: 20) | |
| OTS- | | CM000819.5 | 1777887 | − | TCCGAAGCCT | GCAGCAGGG | − |
| F2 | | Chr8 | | | GAGAAGACC | AGACAGTTCA | |
| | | | | | (SEQ ID | (SEQ ID | |
| | | | | | NO: 21) | NO: 22) | |
| OTS- | | CM000821.5 | 41563383 | + | CCTGACCAGC | CATAATAGG | − |
| F3 | | Chr10 | | | CTGTTTGA | AGTTCCCAGT | |
| | | | | | (SEQ ID | C (SEQ ID | |
| | | | | | NO: 23) | NO: 24) | |
| OTS- | | CM000821.5 | 44919247 | − | AAGCCTGGG | CTTGCGTGGG | − |
| F4 | | Chr10 | | | ACTCCTTAG | TTTAATGT | |
| | | | | | (SEQ ID | (SEQ ID | |
| | | | | | NO: 25) | NO: 26) | |
| OTS- | | CM000825.5 | 101886087 | + | ATCTGCTGAG | TCAATAGCAG | − |
| F5 | | Chr14 | | | CCACAATG | CCCAACCT | |
| | | | | | (SEQ ID | (SEQ ID | |
| | | | | | NO: 27) | NO: 28) | |
| OTS- | | CM000825.5 | 131339903 | + | ATTGGCTTTG | TGCTGTGAGC | − |
| F6 | | Chr14 | | | AAACACTCC | CGAGGTAT | |
| | | | | | (SEQ ID | (SEQ ID | |
| | | | | | NO: 29) | NO: 30) | |
| OTS- | | CM000828.5 | 30987574 | + | CTGGCATTGT | GGAAGGCAG | − |
| F7 | | Chr17 | | | AGCCTGACC | TTACTCAAAG | |
| | | | | | (SEQ ID | A (SEQ ID | |
| | | | | | NO: 31) | NO: 32) | |
| OTS- | | CM000830.5 | 124454124 | + | GGGCAGGAG | TGCAGCGAG | − |
| F8 | | ChrX | | | GACAAGGAG | GACCTGAATG | |
| | | | | | TT (SEQ ID | G (SEQ ID | |
| | | | | | NO: 33) | NO: 34) | |
| OTS- | | AEMK02000 | 183875 | + | ACCATCACTC | CATGTGGGAC | − |
| F9 | | 639.1 | | | CGGGCGACTC | CTCGGATTGA | |
| | | | | | (SEQ ID | (SEQ ID | |
| | | | | | NO: 35) | NO: 36) | |

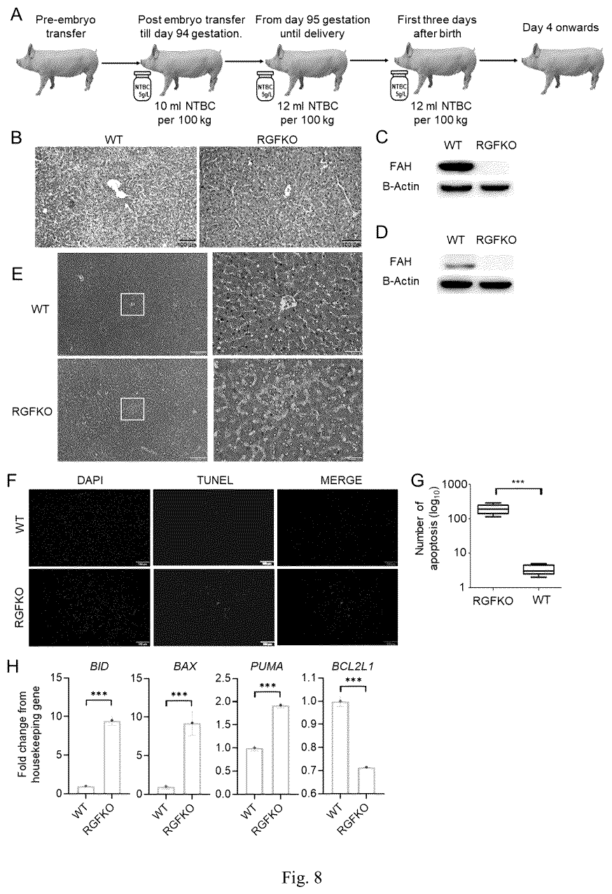

Phenotypic analysis: the 2 RAG2^{-/-}/IL2Rγ^{-/Y}/FAH^{-/-} triple-gene knockout pigs survived 14 days and 29 days respectively (A of FIG. 7). The thymus dysplasia (B of FIG. 7) and the spleen dysplasia (C&D of FIG. 7) of the RAG2^{-/-}/IL2Rγ^{-/Y}/FAH^{-/-} triple-gene edited knockout pigs were observed after pathological dissection. Peripheral blood and spleen PBMC cells of 1 RAG2^{-/-}/IL2Rγ^{-/Y}/FAH^{-/-} triple-gene mutant cloned pig were extracted, and the numbers of mature T cells, B cells, and NK cells were detected by immunological and cytological methods (monocytes labeled by M/G, T cells labeled by CD3 antibody, B cells labeled by CD45RA antibody, and NK cells labeled by CD3 and CD16 antibodies). The results showed that the mature T lymphocytes were significantly reduced and the B lymphocytes were deficient in peripheral blood (E of FIG. 7), the NK cells were deficient in the peripheral blood (D of FIG. 7); considering peripheral B cells were deficient, immature B lymphocytes and immature T cells were retained in the spleen (G of FIG. 7). the mRNA expression levels of the CD8, CD19, IL2Rγ, CD19 genes were significantly reduced in the spleen (H of FIG. 7), the protein expression by the IL2Rγ gene in the spleen was lacked (I of FIG. 7), the immune gene rearrangement played an important role in immune response, and the deficient of IL2Rγ gene function leaded to the loss of TRD, TRB and IGH (VDJ) gene rearrangement function (J of FIG. 7), which indicating that the deficient of immune function was caused by the deficient of IL2Rγ gene function. The FAH gene knockout resulted in the deficient of FAH protein expression in liver (B&C of FIG. 8) and in testis (D of FIG. 8), the liver tissue was severely damaged (E of FIG. 8), the hepatocyte apoptosis was significantly increased (F and G of FIG. 8), and the mRNA expression levels of BID, BAX and PUMA genes were significantly increased in the liver, the mRNA expression levels of BCL2L1 genes anti-apoptosis were significantly decreased (H of FIG. 8). The RAG2^{-/-}/IL2Rγ^{-/Y}/FAH^{-/-} triple-gene knockout dual pig model of severe immunodeficiency and liver injury in the present disclosure effectively removes NK cells in peripheral blood, reduces the occurrence of inflammatory reaction to a certain extent, has a higher humanization degree, and effectively reduces immunological rejection.

### Reversibility Analysis:

the pigs are large mammals. In the process of preparing a liver injury model, it is a whole-new exploration process to maintain tyrosine metabolism on the pigs by using an NTBC drug. The present disclosure firstly obtains the dual pig model of immunodeficiency and liver injury by a CRISPR/Cas9 technology. Besides, after the FAH gene is knocked out, reversibility can be realized by feeding the NTBC drug. If the batch production of the dual pig model of severe immunodeficiency and liver injury is required, the model can be further obtained by a continuous cloning technology, so as to successfully solve the problem of irreversible use of the existing model construction technology.

The present disclosure determined a dosage regimen of the optimum concentration of the NTBC drug (A of FIG. 8) through a large number of preliminary experiments, feeding 10-12 mL 5 g/L of NTBC every 100 kg (A of FIG. 8), and overcame negative effects of insufficient use amount or excessive use amount of the NTBC drug on the RAG2^{-/-}/IL2Rγ^{-/Y}/FAH^{-/-} triple-gene mutant pig (when the use amount was less, the survival rate of the pigs was low, and when the use amount was excessive, the negative effects may be produced on the surrogate sows and piglets) to a certain extent.

In conclusion, the present disclosure provides a method for constructing a dual pig model of severe immunodeficiency and liver injury, and overcomes the problems of long production period, low efficiency, irreversible injury, unsatisfactory humanization degree and the like in the existing model construction technology. The obtained RAG2^{-/-}/IL2Rγ^{-/Y}/FAH^{-/-} triple-gene edited cloned pig shows severe immunodeficiency and liver injury, thymus and spleen dysplasia, reduction of mature T cells, deficiency of B cells and NK cells, increased hepatocyte apoptosis, and high immunodeficiency degree. If the batch production of the pig model of severe immunodeficiency and liver injury can be realized through a continuous cloning technology, the model plays an important effect in the related fields of tumor biology, cell transplantation, humanized animal models and the like, and has a potential market application prospect.

Finally, it is noted that the above-mentioned preferred examples are only used to illustrate rather than limit the technical solutions of the present disclosure. Although the present disclosure has been described in detail with reference to the above-mentioned preferred examples, it will be understood by those skilled in the art that various changes in form and detail may be made therein without departing from the scope of the present disclosure as defined by the claims.

What is claimed is:

1. A RAG2/IL2Rγ FAH triple-gene targeting vector, wherein the targeting vector is an sgRNA expression vector based on a CRISPR/Cas9 system, and an sgRNA comprises a RAG2-sgRNA, an IL2Rγ-sgRNA, and an FAH-sgRNA; and the sgRNA acts on a site located in a coding region of a pig RAG2 gene, a 5th exon of an IL2Rγ gene, and a 2nd exon of an FAH gene; the nucleotide sequence of the RAG2-sgRNA is shown in SEQ ID NO: 1; the nucleotide sequence of the IL2Rγ-sgRNA is shown in SEQ ID NO: 2; and the nucleotide sequence of the FAH-sgRNA is shown in SEQ ID NO: 3.

2. The RAG2/IL2Rγ/FAH triple-gene targeting vector according to claim 1, wherein a framework vector is a pGL3-U6-sgRNA, the number of the framework vector is Addgene no: 51133.

3. A RAG2/IL2Rγ/FAH triple-gene knockout porcine fibroblast cell line, wherein the targeting vector according to claim 1 is transfected into the porcine fetal fibroblast cell line to obtain a targeted positive cell clone, that is the RAG2/IL2Rγ/FAH triple-gene knockout porcine fibroblast cell line or a RAG2^{-/-}/IL2Rγ^{-/Y}/FAH^{-/-} triple-gene edited porcine fibroblast cell line.

4. A method for constructing a dual pig model of severe immunodeficiency and liver injury by using a CRISPR/Cas9 technology, comprising the following specific steps:
(1) construction of RAG2^{-/-}/IL2Rγ^{-/Y}/FAH^{-/-} triple-gene mutation expression vector on the basis of CRISPR/Cas9 system;
aiming at a coding region of a pig RAG2 gene, a 5th exon of an IL2Rγ gene, and a 2nd exon of an FAH gene, designing a targeting sgRNA and connecting same to a framework vector to obtain a pGL3-U6-sgRNA recombinant plasmid; the sgRNA comprises a RAG2-sgRNA, an IL2Rγ-sgRNA, and an FAH-sgRNA; the nucleotide sequence of the RAG2-sgRNA is shown in SEQ ID NO: 1, the nucleotide sequence of the IL2Rγ-sgRNA is shown in SEQ ID NO: 2, and the nucleotide sequence of the FAH-sgRNA is shown in SEQ ID NO: 3;
(2) screening of RAG2^{-/-}/IL2Rγ^{-/Y}/FAH^{-/-} triple-gene edited porcine fibroblast cell line
co-transfecting the pGL3-U6-sgRNA recombinant plasmid and a pST1374-NLS-flag-linker-Cas9 into a porcine fetal fibroblast cell, and obtaining a RAG2^{-/-}/IL2Rγ^{-/Y}/FAH^{-/-} triple-gene edited porcine fibroblast cell line through single cell clone genotype identification and screening;
(3) somatic cell nuclear transfer and embryo transfer
performing a somatic cell nuclear transfer by using the RAG2^{-/-}/IL2Rγ^{-/Y}/FAH^{-/-} triple-gene edited porcine fetal fibroblast cell line as a donor cell to construct a RAG2^{-/-}/IL2Rγ^{-/Y}/FAH^{-/-} triple-gene edited pig cloned embryo, further transplanting the cloned embryo into an oviduct of a surrogate sow, and delivering a RAG2^{-/-}/IL2Rγ^{-/Y}/FAH^{-/-} triple-gene edited cloned pig after 114 days of pregnancy; and
(4) genotype identification and phenotypic analysis of cloned piglet
extracting genomic DNA from a cloned piglet, identifying a RAG2/IL2Rγ/FAH genotype of the cloned piglet by using a molecular biology method, further performing histological and immunohistochemical analysis on organs of thymus, spleen, liver and the like of the cloned pig, and performing immunological and cytological analysis and identification on a mature T cell, a B cell, an NK cell, a hepatocyte and the like to obtain a RAG2^{-/-}/IL2Rγ^{-/Y}/FAH^{-/-} triple-gene edited dual pig model of severe immunodeficiency and liver injury.

5. The method for constructing a dual pig model of severe immunodeficiency and liver injury by using a CRISPR/Cas9 technology according to claim 4, wherein in step 1), the vector is a framework vector pGL3-U6-sgRNA, the number of the framework vector is Addgene no: 51133).

6. The method for constructing a dual pig model of severe immunodeficiency and liver injury by using a CRISPR/Cas9 technology according to claim 4, wherein in step 2), the transfection method comprises lipofection transfection and/or nuclear transfection; and in step 3), the donor cell can be a RAG2^{-/-}/IL2Rγ^{-/Y}/FAH^{-/-} triple-gene edited single cell clone or a cloned fetal fibroblast and a cloned porcine fibroblast cell.

7. The method for constructing a dual pig model of severe immunodeficiency and liver injury by using a CRISPR/Cas9 technology according to claim 6, the transfection method is nuclear transfection.

8. The method for constructing a dual pig model of severe immunodeficiency and liver injury by using a CRISPR/Cas9 technology according to claim 4, wherein in step 3), batch production of the RAG2^{-/-}/IL2Rγ^{-/Y}/FAH^{-/-} triple-gene edited cloned pig is required, the pig can be obtained by a continuous cloning technology.

9. Use of the targeting vector according to claim 1 in constructing a dual pig model of severe immunodeficiency and liver injury.

10. Use of the porcine fibroblast cell line according to claim 3 in constructing a dual pig model of severe immunodeficiency and liver injury.

11. Use of method according to claim 4 in constructing a dual pig model of severe immunodeficiency and liver injury.
